(19) United States
(12) Patent Application Publication
(10) Pub. No.: US 2008/0132451 A1
(43) Pub. Date: Jun. 5, 2008

(54) MODULATION OF POLYSIALYLATED NEURAL ADHESION MOLECULES (PSA-NCAM) AS A REGULATOR OF OCULAR DISEASE

(75) Inventors: Martha E. Gadd, Fort Worth, TX (US); Gustav Graff, Cleburne, TX (US)

Correspondence Address: ALCON, IP LEGAL, TB4-8, 6201 SOUTH FREEWAY, FORT WORTH, TX, 76134, US

(73) Assignee: Alcon Manufacturing Ltd., Fort Worth, TX (US)

(21) Appl. No.: 11/947,067

(22) Filed: Nov. 29, 2007

Related U.S. Application Data
(60) Provisional application No. 60/873,418, filed on Dec. 1, 2006.

(51) Int. Cl. A61K 38/18; A61P 27/02; A61K 38/30

(52) U.S. Cl. 514/12

(57) ABSTRACT

A method of treating dry eye or dry eye symptom includes topically administering a formulation to the eye. The formulation includes an ophthalmically acceptable carrier and a pharmaceutically effective amount of a positive modulator of PSA-NCAM. The administered formulation treats dry eye or dry eye symptoms. A method of stimulating the expression of PSA-NCAM in neural beds of an eye includes topically administering a formulation to the eye. The formulation includes an ophthalmically acceptable carrier and a pharmaceutically effective amount of a positive modulator of PSA-NCAM. The administered formulation may stimulate the expression of PSA-NCAM in the neural beds of the eye. A formulation includes an ophthalmically acceptable carrier and a therapeutically effective amount of a positive modulator of polysialic acid-neural cell adhesion molecule (PSA-NCAM).

This application claims priority to U.S. Provisional Application, U.S. Ser. No. 60/873,418 filed Dec. 1, 2006.

### FIELD OF THE INVENTION

This present invention relates generally to methods of treatment for dry eye.

### BACKGROUND OF THE INVENTION

Dry eye, also known generically as keratoconjunctivitis sicca, is a common opthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and foreign body sensation of the eyes. In severe cases, vision may be substantially impaired. Other diseases, such as Sjogren's disease, several autoimmune conditions such a rheumatoid arthritis and lupus and cicatricial pemphigoid manifest dry eye complications. Finally, trauma to the eye during surgical procedures such as lasik surgery or even photorefractive keratectomy may lead to dry eye symptoms.

Although it appears that dry eye may result from a number of unrelated pathogenic causes, all presentations of the complication share the common effect of the breakdown of the pre-ocular tear film, which results in damage of the exposed outer surface and many of the symptoms outlined above.

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye. Another approach involves the provision of lubricating substances in lieu of artificial tears.

Although these approaches have met with some success, problems in the treatment of dry eye nevertheless remain. The use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive. Transient symptoms of dry eye associated with refractive surgery have been reported to last in some cases from six weeks to six months or more following surgery.

Aside from efforts directed primarily to the alleviation of symptoms associated with dry eye, methods and compositions directed to treatment of the dry eye condition have also been pursued. Many of these compositions use steroids which have limited utility due to concomitant increases in intraocular pressure and/or development of cataracts in the eye.

Thus, there is a continuing need for an effective pharmaceutical intervention to treat the underlying causes of dry eye. An understanding of the morphological changes associated with dry eye provides a starting point for the development of appropriate pharmaceutical treatments. Analysis of the corneas of various patient populations with dry eye or dry eye symptoms show abnormalities in neuronal beds as imaged with confocal microscopy and also show abnormalities in corneal sensitivity. The most marked change in the nerve beds is the appearance of tortuosities. A pharmaceutical formulation that may address these abnormalities in dry eye patients is, therefore, a worthwhile pursuit.

### SUMMARY OF THE INVENTION

In some aspects, embodiments of the present invention relate to a method of treating dry eye or dry eye symptoms. The method includes topically administering a formulation to the eye, wherein the formulation includes an ophthalmically acceptable carrier and a pharmaceutically effective amount of a positive modulator of PSA-NCAM. The administered formulation treats dry eye or dry eye symptoms.

In other aspects, embodiments of the present invention relate to a method of stimulating the expression of PSA-NCAM in neural beds of an eye. The method includes topically administering a formulation to the eye. The formulation includes an ophthalmically acceptable carrier and a pharmaceutically effective amount of a positive modulator of PSA-NCAM. The administered formulation stimulates the expression of PSA-NCAM in the neural beds of the eye.

In yet another aspect, embodiments of the present invention provide a formulation comprising an ophthalmically acceptable carrier and a therapeutically effective amount of a positive modulator of polysialic acid-neural cell adhesion molecule (PSA-NCAM).

Advantageously, the present invention provides a formulation and method for the treatment of dry eye that addresses the need for repair of the neuronal bed. Such an approach may obviate the need for costly long term use of tear replacement formulations.

### BRIEF DESCRIPTION OF THE DRAWINGS

The foregoing and other features and aspects of the present invention will be best understood with reference to the following detailed description of a specific embodiment of the invention, when read in conjunction with the accompanying drawings, wherein:

FIGS. 1a and 1b show NCAM homophilic binding and PSA-NCAM homophilic binding, respectively.

### DESCRIPTION OF THE INVENTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Referring to the drawings in general, it will be understood that the illustrations are for the purpose of describing a particular embodiment of the invention and are not intended to limit the invention thereto.

In some embodiments, a method of treating dry eye or dry eye symptoms may include topically administering a formulation to the eye. The formulation may include an ophthalmically acceptable carrier and a pharmaceutically effective amount of a positive modulator of polysialic acid-neural cell adhesion molecule (PSA-NCAM). The administration of the formulation to the patient with dry eye with may treat the underlying cause of dry eye.

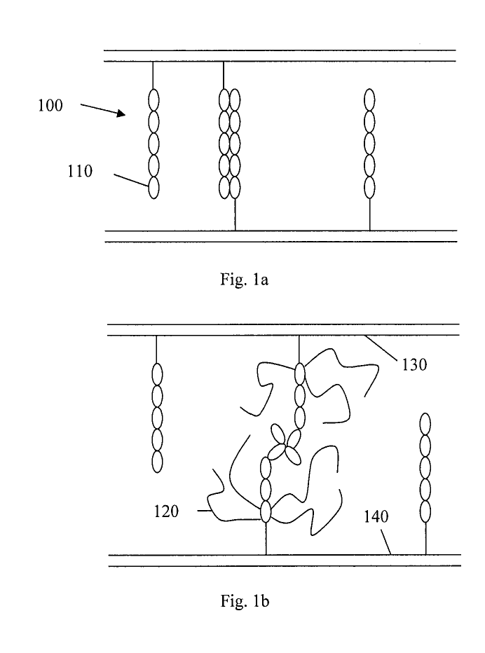

Neural cell adhesion molecule (NCAM) and its polysialylated form (PSA-NCAM) may regulate the interactions between neuronal cells and other neighboring cells, as shown in FIG. 1a and FIG. 1b (adapted from Kiss et al. Brain Research Reviews, 2001 (36), 175-184.). FIG. 1a shows a hypothetical homophilic binding of NCAM 100 that may stabilize cell-cell contacts through the interaction of five immunoglobulin (Ig) domains 110 within NCAM 100. As shown in FIG. 1b, the presence of polysialic acid (PSA) 120 on NCAM 100 may lead to reduced interactions of the Ig domains, which may reduce adhesion between neighboring cell membranes 130 and 140. It has also been suggested that the presence of PSA 120 on NCAM 100 may act to alter intracellular signaling pathways. Importantly, homophilic binding with NCAM and homophilic binding with PSA-NCAM may modulate neurite outgrowth, each in a different manner.

The “relaxed” cell-cell interaction associated with cells expressing surface PSA-NCAM may provide a more open environment for neurite growth facilitating branching, for example. Indeed, PSA-NCAM has been associated with neuronal plasticity and the growth of neurons. PSA-NCAM expression may be elevated during development and after a lesion (e.g. after lasik surgery). Regulation of PSA-NCAM on the cellular surface may be controlled by sialyltransferases that are responsible for the post-translational modification and by intracellular trafficking that dictates its membrane-associated residency time.

Thus, embodiments of the present invention include treating dry eye by activating PSA-NCAM as a modulating protein for nerve bed plasticity within the cornea. The biosynthesis and cell surface expression of PSA-NCAM may be affected by a large number of complex cell signaling pathways, the sialyltranserferase enzymes that decorate NCAM at the Golgi apparatus, and a number of growth factors involved in neural regeneration.

In some embodiments, a positive modulator of PSA-NCAM is a growth factor. A number of growth factors may spur neuronal growth. In some embodiments, a growth factor is used to stimulate expression of PSA-NCAM on the cell surface leading to enhanced neurite outgrowth.

In some embodiments, the positive modulator of PSA-NCAM is a growth factor such as nerve growth factor (NGF). NGF is known to be important for the survival and maintenance of sympathetic and sensory neurons. NGF's receptor TrkA is a protein tyrosine kinase (PTK). These kinases have been tied to cellular uptake of calcium, which in turn is associated with neurite outgrowth. NGF, as disclosed herein, encompasses natural, synthetic and recombinant forms of NGF, and may include those having deleted, replaced or altered amino acid sequences in comparison with the full-length natural NGF polypeptide or biologically active fragments thereof.

In some embodiments, the positive modulator of PSA-NCAM is an epidermal growth factor (EGF). EGF is a growth factor that plays an important role in the regulation of cell growth, proliferation and differentiation. EGF acts by binding with high affinity to epidermal growth factor receptor (EGFR) on the cell surface and stimulating the intrinsic protein-tyrosine kinase activity of the receptor. The tyrosine kinase activity in turn initiates a signal transduction cascade which results in a variety of biochemical changes within the cell including a rise in intracellular calcium levels. The rise in calcium levels may induce expression of PSA-NCAM since increased calcium levels are associated with neurite outgrowth. EGF, as disclosed herein, encompasses natural, synthetic and recombinant forms of EGF, and may include those having deleted, replaced or altered amino acid sequences in comparison with the full-length natural EGF polypeptide or biologically active fragments thereof.

In alternate embodiments, the positive modulator of PSA-NCAM is an insulin-like growth factor (IGF), and may include, but are not limited to IGF-I and IGF-II. IGF-I and IGF-II, as disclosed herein, encompasses natural, synthetic and recombinant forms, and may include those having deleted, replaced or altered amino acid sequences in comparison with the full-length natural polypeptide or biologically active fragments thereof.

Other growth factors which may be utilized in the present invention include transforming growth factor-alpha (TGF-α), keratinocyte growth factor (KGF), platelet-derived growth factors (PDGF-BB, -AA, or -AB), basic fibroblast growth factor (b-FGF), acid fibroblast growth factor (a-FGF), angiogenin, and other proteins or polypeptides having mitogenic receptors relative to neural cells. As disclosed herein, these proteins or polypeptides encompass natural, synthetic and recombinant forms, and include those having deleted, replaced or altered amino acid sequences in comparison with the full-length natural protein or polypeptide or biologically active fragments thereof.

The growth factors used in the present invention may be human derived. As used herein, the term “human derived” encompasses substrates recovered from human tissues and substrates produced from human cell lines by means of recombinant DNA technology.

In yet another embodiment, aberrant PSA-NCAM is rescued by the use of a neurotrophic factor, for example brain-derived neurotrophic factor, BDNF. BDNF may help support the survival of existing neurons, and encourage the growth and differentiation of new neurons. It has been shown that PSA-NCAM is expressed in injured brain tissue where tissue repair may benefit from reorganization and plasticity. The neurotrophin family (including NGF) also includes the growth factors neurotrophin 3 (NT-3), and neurotrophin 4 (NT-4). Each of these may also be a positive modulator of PSA-NCAM. BDNF, NT-3, and NT-4, as disclosed herein, encompasses natural, synthetic and recombinant forms of these neurotrophins, and include those having deleted, replaced or altered amino acid sequences in comparison with the full-length natural neurotrophin polypeptide or biologically active fragments thereof.

In some embodiments, a method of treating dry eye includes topically administering to the eye a composition that comprises an ophthalmically acceptable carrier and a pharmaceutically effective amount of a modulator of PSA-NCAM.

In some embodiments, the pharmaceutically effective amount of the positive modulator of PSA-NCAM ranges from 2 ng/mL to 3 μg/mL.

For such topical administration, the compositions administered may also include various other ingredients as carriers, including but not limited to surfactants, tonicity substrates, buffers, preservatives, co-solvents and viscosity building substrates.

Various tonicity substrates may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity substrate will vary, depending on the particular substrate to be added. In general, however, the compositions will have a tonicity substrate in an amount sufficient to cause the final composition to have an ophthalmically acceptable osmolarity (generally about 150-450 mOsm/L, preferably 250-350 mOsm/L).

An appropriate buffer system (e.g., sodium phosphate, sodium acetate, sodium citrate, sodium borate or boric acid) may be added to the compositions to prevent pH drift under storage conditions. The particular concentration will vary, depending on the substrate employed. Preferably, however, the buffer will be chosen to maintain a target pH within the range of pH 6-7.5.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; and (ii) are safe. Examples of artificial tears compositions useful as artificial tears carriers include, but are not limited to, commercial products, such as Tears Naturale®, Tears Naturale II®, Tears Naturale Free®, and Bion Tears® (Alcon Laboratories, Inc., Fort Worth, Tex.). Examples of phospholipid carrier formulations include those disclosed in U.S. Pat. Nos. 4,804,539 (Guo et al.), 4,883,658 (Holly), 4,914,088 (Glonek), 5,075,104 (Gressel et al.), 5,278,151 (Korb et al.), 5,294,607 (Glonek et al.), 5,371,108 (Korb et al.), 5,578,586 (Glonek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear integrity, or otherwise provide temporary relief of dry eye symptoms and conditions upon topical administration to the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

In some embodiments, other compounds may also be added to the ophthalmic compositions to increase the viscosity or enhance the physical stability of the composition. Examples of viscosity enhancing substrates include, but are not limited to: polysaccharides, such as hyaluronic acid and its salts, chondroitin sulfate and its salts, dextrans, various polymers of the cellulose family; carboxy vinyl polymers such as carbomers (e.g., carbomer 974P); and acrylic acid polymers. In general, the phospholipid carrier or artificial tears carrier compositions will exhibit a viscosity of 1 to 400 centipoises (“cps”).

Topical ophthalmic products are typically packaged in multidose form. Preservatives may be beneficial to prevent microbial contamination during use. Suitable preservatives include: chlorobutanol, benzododecinium bromide, methyl paraben, propyl paraben, phenylethyl alcohol, edetate disodium, sorbic acid, polyquaternium-1, or other substrates known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, 1-2 drops of such compositions will be administered from once to many times per day.

All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed:

1. A method of treating dry eye or dry eye symptoms comprising:
topically administering a formulation to the eye, the formulation comprising:
an ophthalmically acceptable carrier; and
a pharmaceutically effective amount of a positive modulator of PSA-NCAM.

2. The method of claim 1, wherein the positive modulator of PSA-NCAM is a growth factor.

3. The method of claim 2, wherein the growth factor is a nerve growth factor (NGF).

4. The method of claim 2, wherein growth factor is an epidermal growth factor (EGF).

5. The method of claim 2, wherein the growth is an insulin-like growth factor (IGF).

6. The method of claim 1, wherein the modulator of PSA-NCAM is a neurotrophic factor.

7. The method of claim 6, wherein the neurotrophic factor is selected from BDNF, NT-3 and NT-4.

8. The method of claim 1, wherein the pharmaceutically effective amount of the positive modulator of PSA-NCAM is 2 ng/mL to 3 μg/mL.

9. A method of stimulating the expression of PSA-NCAM in neural beds of an eye comprising:
topically administering a formulation to the eye, the formulation comprising:
an ophthalmically acceptable carrier; and
a pharmaceutically effective amount of a positive modulator of PSA-NCAM.

10. The method of claim 9, wherein the positive modulator of PSA-NCAM is a growth factor.

11. The method of claim 10, wherein the growth factor is selected from the group consisting of a neural growth factor (NGF); an epidermal growth factor (EGF); and an insulin-like growth factor (IGF).

12. The method of claim 9, wherein the modulator of PSA-NCAM is a neurotrophic factor.

13. A formulation comprising an ophthalmically acceptable carrier and a therapeutically effective amount of a positive modulator of polysialic acid-neural cell adhesion molecule (PSA-NCAM).

14. The formulation of claim 13, wherein the modulator of PSA-NCAM is a growth factor.

15. The formulation of claim 14, wherein the growth factor is nerve growth factor (NGF).

16. The formulation of claim 14, wherein the growth factor is an epidermal growth factor (EGF).

17. The formulation of claim 14, wherein the growth factor receptor is an insulin-like growth factor (IGF).

18. The formulation of claim 13, wherein the modulator of PSA-NCAM is a neurotrophic factor.

19. The formulation of claim 13, wherein the pharmaceutically effective amount of the positive modulator of PSA-NCAM is 2 ng/mL to 3 μg/mL.
